SEQUENCE LISTING (2 sequences)

SEQ ID NO: 1 - DNA, 2903 bp, Artificial Sequence
  synthetic polynucleotide
ttgacagctagctcagtcctagggattgtgctagcccatgggagttccccgcgccagcggggataaaccgtgagacctctagaggtctcagagttccccgcgccagcggggataaaccgcagctcccattttcaaacccatcaagacgcggtaccctcgagtctggtaaagaaaccgctgctgcgaaatttgaacgccagcacatggactcgtctactagcgcagcttaattaacctaggctgctgccaccgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggggatctcgacgctctcccttatgcgactcctgcattaggaaat

SEQ ID NO: 2 - DNA, 301 bp, Artificial sequence
  synthetic polynucleotide
gaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacaattgtgagcgctcacaattactgtttctccaattgtgagcgctcacaatt

(19) United States
(12) Patent Application Publication
(10) Pub. No.: US 2015/0315576 A1
(43) Pub. Date: Nov. 5, 2015

(54) GENETIC DEVICE FOR THE CONTROLLED DESTRUCTION OF DNA

(71) Applicant: MASSACHUSETTS INSTITUTE OF TECHNOLOGY, Cambridge, MA (US)

(72) Inventors: Brian Caliando, Cambridge, MA (US); Christopher Voigt, Belmont, MA (US)

(73) Assignee: Massachusetts Institute of Technology, Cambridge, MA (US)

(21) Appl. No.: 14/440,166

(22) PCT Filed: Nov. 1, 2013
(86) PCT No.: PCT/US13/68139

§ 371 Date: May 1, 2015

Related U.S. Application Data
(60) Provisional application No. 61/721,103, filed on Nov. 1, 2012.

(51) Int. Cl. C12N 15/113

(52) U.S. Cl. CPC C12N 15/113; C12N 2310/14

(57) ABSTRACT

The invention relates to DNA destruction devices and related methods reagents and kits. The DNA destruction devices are useful for achieving target specific DNA destruction in vivo using a system that involves an actuator element and a CRISPR array, under specific regulatory control.

### RELATED APPLICATIONS

This application claims priority under 35 U.S.C. §119(e) to U.S. Provisional Application Ser. No. 61/721,103, entitled “GENETIC DEVICE FOR THE CONTROLLED DESTRUCTION OF DNA” filed on Nov. 1, 2012, which is herein incorporated by reference in its entirety.

### FEDERALLY SPONSORED RESEARCH

This invention was made with government support under Grant No. EECO540879 awarded by the National Science Foundation and under Contract No. N66001-12-C-4187 awarded by the Space and Naval Warfare Systems Center. The United States government has certain rights in the invention.

### BACKGROUND OF THE INVENTION

Genetically engineered microbial biocatalysts are becoming increasingly valuable platforms for the production of industrially, medicinally, and nutritionally relevant biomolecules. As the scope and complexity of these biocatalysts increase, so does the need for more precise and reliable means of controlling the host organism's biochemistry.

CRISPR interference is a recently discovered facet of prokaryotic biology present across many phyla of archea and eubacteria. Somewhat similar to the more familiar eukaryotic RNAi machinery, the CRISPR machinery acts as part of the host's immune system defending the cell from viruses, transposons, and plasmids by using short, genetically-encoded RNA guide strands to target matching nucleotide sequences for destruction via predictable base-pair interactions. Unlike RNAi, however, many CRISPR systems are designed to target and degrade DNA rather than RNA.

### SUMMARY OF THE INVENTION

The invention, in various aspects, relates to synthetic DNA destruction devices and related methods, reagents, kits and compositions.

Aspect of the invention relate to a synthetic DNA destruction device (DDD) that includes a nucleic acid sequence having an actuator sequence under the control of a first regulatory element and a nucleic acid sequence having a Clustered Regular Interspaced Short Palindromic Repeats (CRISPR) array under the control of a second regulatory element. In some embodiments, the nucleic acid sequence having an actuator sequence and the nucleic acid sequence having a CRISPR array are linked.

In some embodiments, the actuator sequence encodes a DNA targeting/degradation protein. In some embodiments, the actuator sequence encodes 2-10 DNA targeting/degradation proteins. In some embodiments, the actuator sequence includes a CRISPR-associated (cas) gene. In some embodiments, the cas gene is selected from the group consisting of cas3 and casABCDE. In some embodiments, the cas gene is six cas genes: cas3 and casABCDE. In other embodiments, the cas gene is a single cas gene.

In some embodiments, the CRISPR array includes interspersed sets of target specific spacer sequences between palindromic repeat sequences. In some embodiments, the interspersed sets of target specific spacer sequences are 29-33 base pairs in length. In some embodiments, the interspersed sets of target specific spacer sequences are 30-33 base pairs in length. In some embodiments, the interspersed sets of target specific spacer sequences are 32 base pairs in length. In some embodiments, the palindromic repeat sequences are identical to one another. In some embodiments, the palindromic repeat sequences are 29 nucleotides in length.

In some embodiments, the target specific spacer sequences are complementary to a target sequence. In some embodiments, the target specific spacer sequences are 100% complementary to a target sequence. In some embodiments, the first 5-15 nucleotides of the target specific spacer sequences have 100% complementary to a target sequence. In some embodiments, the first 10 nucleotides of the target specific spacer sequences have 100% complementary to a target sequence. In some embodiments, the target specific spacer sequences are at least 85% complementary to a target sequence.

In some embodiments, the target specific spacer sequences have an adjacent discriminator sequence. In some embodiments, the discriminator sequence is 5′ of the target specific spacer sequences. In other embodiments, the discriminator sequence is 3′ of the target specific spacer sequences. In some embodiments, the adjacent discriminator sequence is a PAM sequence. In some embodiments, the PAM sequence is 3 base pairs in length. In some embodiments, the presence of the PAM sequence adjacent to the target sequence allows degradation of the nucleic acid containing the target sequence. In other embodiments, the adjacent discriminator sequence is a non-inhibitory sequence. In some embodiments, the presence of the non-inhibitory sequence allows degradation of the target nucleic acid. In some embodiments, the PAM has a sequence selected from the group consisting of ATG, AAG, GAG, AGG, AAA, AAC, AAT, TAG, TTG, ATA, CAG, TGG, GTG, GGG, AND TAA .

In some embodiments, the target sequence is part of any one or more of a DNA based transposon, bacteriophage nucleic acid, plasmid, and/or chromosome.

In some embodiments, wherein the first regulatory element is a first inducible promoter. In some embodiments, the second regulatory element is a second inducible promoter. In some embodiments, the first regulatory element is an activation element that induces expression of the actuator sequence in response to one or more activation signals. In some embodiments, wherein the second regulatory element is a second activation element that induces the production of a DNA interference RNA from the CRISPR array in the presence of an activation signal. In some embodiments, the activation signal is a chemical signal. In some embodiments, the chemical signal is arabinose. In other embodiments, the activation signal is an environmental signal.

In some embodiments, the first regulatory element is an inhibitory element that maintains the actuator in an inactive state by the presence of an inhibitory signal. In some embodiments, the inhibitory signal is a chemical signal. In other embodiments, wherein the inhibitory signal is an environmental signal.

In some embodiments, the first regulatory element is an inhibitory element and an activation element. In some embodiments, the second regulatory element is an inhibitory element and an activation element.

In some embodiments, the second regulatory element is an inhibitory element that maintains the CRISPR array in an inactive state by the presence of an inhibitory signal. In some embodiments, the inhibitory signal is an environmental signal. In some embodiments, the environmental signal is glucose.

In some embodiments, the DDD further includes a processing element. In some embodiments, the processing element is a tracrRNA.

In some embodiments, DDD is targeted to destroy DNA. In some embodiments, the DDD is targeted to destroy RNA.

Aspects of the invention relate to a kit including one or more containers housing one or more components of a synthetic DNA destruction device (DDD) selected from a nucleic acid sequence having an actuator sequence under the control of a first regulatory element and a nucleic acid sequence having a Clustered Regular Interspaced Short Palindromic Repeats (CRISPR) array under the control of a second regulatory element, and instructions for delivering the components to a living cell. In some embodiments, the kit includes a single nucleic acid sequence having both the actuator sequence under the control of the first regulatory element and the CRISPR array under the control of the second regulatory element. In some embodiments, the kit includes separate single nucleic acid sequences for the actuator sequence under the control of the first regulatory element and the CRISPR array under the control of the second regulatory element.

In some embodiments, the nucleic acid sequences for the actuator sequence is a plasmid. In some embodiments, the nucleic acid sequences for the CRISPR array is a plasmid. In some embodiments, the kit further includes an activation signal compound. In some embodiments, the kit further includes an inhibitory signal compound.

In some embodiments, the synthetic DDD is a synthetic DDD as described herein.

In some embodiments, the nucleic acid sequence having the CRISPR array has at least two palindromic repeat sequences with a spacer region positioned between the at least two palindromic repeat sequences, wherein the spacer region includes at least two restriction enzyme sequences. In some embodiments, the kit includes a container housing a restriction enzyme, wherein the restriction enzyme is capable of cleaving at least one of the restriction enzyme sequences.

In some embodiments, the nucleic acid sequence having the CRISPR array has at least four palindromic repeat sequences with spacer regions positioned between each of the palindromic repeat sequences.

In some embodiments, the kit further includes bacteriophage particles.

Aspects of the invention relate to a method for destroying target specific DNA in a living host cell, involving contacting a living modified host cell having an exogenous nucleic acid actuator sequence under the control of a first regulatory element and an exogenous target specific nucleic acid Clustered Regular Interspaced Short Palindromic Repeats (CRISPR) array under the control of a second regulatory element with a first regulatory signal, wherein the first regulatory signal induces the expression of the actuator sequence to produce an actuator protein, wherein the cell is exposed to a second regulatory signal and the second regulatory signal induces the production of a target specific DNA interference RNA, and wherein the actuator protein and the target specific DNA interference RNA destroy a target DNA in the host cell.

In some embodiments, the actuator protein is a complex of proteins.

In some embodiments, the method involves contacting the host cell with the second regulatory signal. In some embodiments, the method further comprises identifying one or more target nucleic acid sequences in a host cell for destruction. In some embodiments, the method further comprises contacting a host cell with the exogenous nucleic acid actuator sequence and the exogenous target specific nucleic acid CRISPR array to create the modified host cell. In some embodiments, wherein the modified host cell is a prokaryotic cell. In some embodiments, the modified is a bacterial cell. In some embodiments, the bacterial cell is a Escherichia coli cell. In some embodiments, the modified host cell contains an exogenous confidential or dangerous target DNA sequence.

In some embodiments, at least one of the first or second regulatory signals is an environmental signal.

In some embodiments, the target specific DNA is a synthetic plasmid DNA encoding specific sets of genetic regulatory elements and wherein the target specific DNA is triggered for destruction under specific conditions.

In some embodiments, at least one of the first or second regulatory signals is an environmental signal.

In some embodiments, the host cell is being manipulated during an industrial fermentation and wherein the environmental signal triggers target specific DNA destruction in order to alter the hosts downstream expression capabilities. In some embodiments, the target DNA is host cell DNA.

In some embodiments, the method involves contacting the living modified host cell with synthetic DDD as described herein.

In some embodiments, the exogenous nucleic acid actuator sequence and the exogenous target specific nucleic acid CRISPR array are contained on the same nucleic acid molecule. In some embodiments, the exogenous nucleic acid molecule is a plasmid. In other embodiments, the exogenous nucleic acid actuator sequence is in a chromosome. In some embodiments, the exogenous target specific nucleic acid CRISPR is in a chromosome. In some embodiments, the exogenous nucleic acid actuator sequence and the exogenous target specific nucleic acid CRISPR array are contained on different nucleic acid molecules. In some embodiments, the exogenous nucleic acid molecules are plasmids.

Aspects of the invention relate to a plasmid comprising a nucleic acid sequence having a Clustered Regular Interspaced Short Palindromic Repeats (CRISPR) array under the control of a regulatory element, wherein the nucleic acid sequence having the CRISPR array has at least two palindromic repeat sequences with a spacer region positioned between the at least two palindromic repeat sequences, wherein the spacer region includes at least two restriction enzyme sequences and a nucleic acid sequence encoding a selectable marker.

In some embodiments, the regulatory element is a constitutive promoter. In other embodiments, the regulatory element is an inducible promoter. In some embodiments, the restriction enzyme sequences are BsaI.

In some embodiments, the nucleic acid sequence having the CRISPR array has at least four palindromic repeat sequences with spacer regions positioned between each of the palindromic repeat sequences. In some embodiments, one or more double-stranded synthetic oligonucleotides is ligated between the repeat sequences. In some embodiments, the plasmid has a nucleotide sequence having at least 80% complementarity with SEQ ID NO. 1. In some embodiments, the plasmid has a nucleotide sequence having at least 90% complementarity with SEQ ID NO. 1. In other embodiments, the plasmid has a nucleotide sequence of SEQ ID NO. 1.

Each of the limitations of the invention can encompass various embodiments of the invention. It is, therefore, anticipated that each of the limitations of the invention involving any one element or combinations of elements can be included in each aspect of the invention.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The figures are illustrative only and are not required for enablement of the invention disclosed herein.

FIG. 1 schematically presents a the DNA destruction device (DDD) in the context of an E. coli cell.

FIG. 2 is a set of drawings depicting various synthetic constructs. FIG. 2A shows an exemplary synthetic CRISPR array spacer sequence containing Spacer Z, which is target to the 3′ end of the wild-type bla gene. FIG. 2B shows a native DNA sequence taken from the wild-type bla gene that contains both a proto-spacer identical to Spacer Z and an endogenous 5′ AAG PAM. This sequence will be targeted for degradation by a device containing the Spacer Z sequence. FIG. 2C depicts a synthetic mutated DNA sequence, bla K252G, that evade targeting by Spacer Z because it contains an inactivating mutation in the essential 5′ PAM sequence. FIG. 2D presents a synthetic mutated bla DNA sequence that evades targeting because the proto-spacer is no longer identical to Spacer Z. Flanking CRISPR repeat sequences are highlighted in blue, and Spacer Z sequence is given in larger font. Proto-spacer sequence identical to Spacer Z is underlined, and mismatches are highlighted in red. Functional PAM positions (5′ AWG) are highlighted in pink, non-functional in green.

FIG. 3 is a schematic and a bar graph. FIG. 3A depicts a schematic of the experimental design to assess the DDD degrading an existing target DNA. FIG. 3B presents a time-course for the loss of the ampicillin resistance-conferring plasmid.

FIG. 4 is a set of drawings. FIG. 4A depicts a schematic of the experimental design to assess the DDD blocking entry of target DNA. FIG. 4B shows the blocking efficiency of several DDD actuator variants.

FIG. 5 is an evaluation of 64 possible 3 base pair PAM sequences using the assay described in FIG. 4A.

FIG. 6 is a schematic and a graph. FIG. 6A presents a diagram for evaluating CRISPR interference of a DNA molecule that is entering the host cell. FIG. 6B shows results of an experiment assessing the ability of host cells containing plasmid-targeting or plasmid-non-targeting CRISPR/cas systems to degrade entering nucleic acid.

FIG. 7 presents an exemplary vector engineered for easy insertion of target spacer sequences.

FIG. 8 schematically presents a method for Scarless Type II-S cloning for the integration of new spacer sequences (Engler C, Gruetzner R, Kandzia R, Marillonnet S (2009) Golden Gate Shuffling: A One-Pot DNA Shuffling Method Based on Type IIs Restriction Enzymes. PLoS ONE 4(5): e5553).

FIG. 9 shows three exemplary promoter constructions including a L-arabinose/IPTG-activating promoter, a L-arabinose-activating, glucose-repressing promoter; and a dual L-arabinose/IPTG-activating, glucose-repressing promoter.

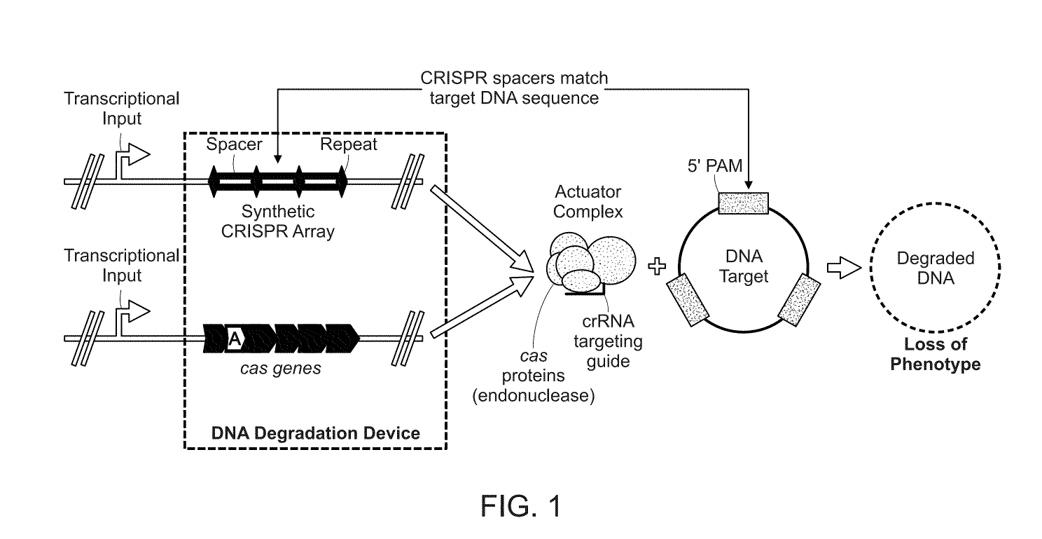

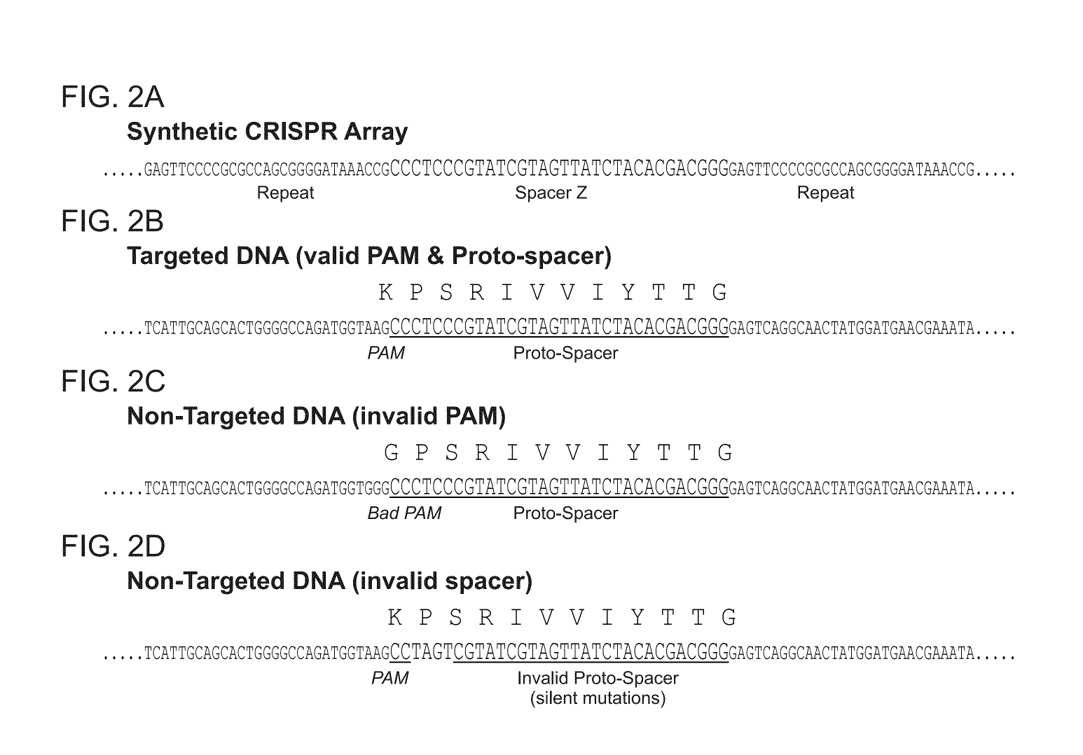

### DETAILED DESCRIPTION

The invention involves, in some aspects, the utilization of unique features of CRISPR biology. Using the CRISPR system, a DNA destruction device (DDD) has been generated that enables target specific DNA and in some instances, RNA destruction in a highly controlled and regulated manner. In response to a chemical input signal, the DDD targets and destroys user-specified native and/or synthetic DNA sequences resulting in the host's loss of both genotype and associated phenotype. The device is highly effective at both removing stable plasmids from cells and causing cell death when targeted to the host genome. Furthermore, DDD degradation renders the target sequence information more difficult to recover by PCR. In this manner, the DDD serves as a “DELETE” key that can be implemented to act on any or all elements of a biocatalyst's synthetic genetic program, thereby offering the user greater control over a broad range of host metabolic and biochemical functionality.

The DDDs of the invention have a number of novel uses. A DDD or Genetic device that can degrade specific pieces of DNA in vivo in response to a user defined signal may serve as a generic tool for the construction of various novel and improved genetic programs for controlling microbial biocatalysts. For example, the devices of the invention provide the user with the ability to delete a very specific target DNA at a specific time or under specific circumstances. This allows the user to fine tune the cellular regulation processes at the DNA level with precision. Additionally, these devices can be used to manipulate microorganisms to regulate potentially hazardous DNA sequences therein. For example, a microorganism which includes a DNA encoding an infectious agent or other toxic agent may be designed to include a DDD at least some components of which are repressed until exposed to an environmental trigger. If the microorganism is accidentally released into the environment, or is going to be disposed of and encounters the environmental signal, then the specific DNA will be destroyed and the potential hazard contained. The same scenario could be achieved using an activation signal instead or in addition to a repressor, as described in more detail below.

Thus, in some aspects a synthetic DDD is made up of a nucleic acid sequence having an actuator sequence under the control of a first regulatory element and a nucleic acid sequence having a Clustered Regular Interspaced Short Palindromic Repeats (CRISPR) array under the control of a second regulatory element. The two nucleic acids may be parts of the same nucleic acid sequence. For instance, they may be both included in a plasmid that has the two nucleic acids linked together by nucleotides, optionally with a number of nucleotides in between. Alternatively the two nucleic acids may be physically separated from one another. For instance, each may be present on a separate discreet plasmid.

The DDD has two main components, an actuator sequence and a CRISPR array. The actuator sequence typically encodes one or more DNA targeting/degradation proteins. For instance it may encode 1, 2-10, 2-15, 2-20, 3-10, 3-15, 3-20, 4-5, 4-10, 4-15, 4-20, 5-10, 5-15, 5-20, 10-15, 10-20, or more DNA targeting/degradation proteins. These actuator proteins, alone or assembled in a complex or otherwise working together, function to destroy DNA that has been marked for destruction by the CRISPR array. In some embodiments the actuator sequence is a CRISPR-associated (cas) gene, such as, for instance, cas3 and/or casABCDE.

The CRISPR array may have a classical CRISPR array structure. For instance, the DDD may include interspersed sets of target specific spacer sequences between palindromic repeat sequences. Often the CRISPR array includes one more palindromic repeat sequence than the target specific spacer sequence such that the target specific spacer sequences are interspersed between the repeat sequences. A palindromic repeat sequence is a consensus CRISPR repeat. Consensus CRISPR repeat typically has multiple short direct repeats, which show no or very little sequence variation within a given CRISPR locus. These sequences are well known in the art.

The naturally occurring E coli CRISPR repeat sequence is the following (29 bp): 5′-GAGTTCCCCGCGCCAGCGGGGATAAACCG-3′ (SEQ ID NO. 3).

The CRISPR repeat sequence of the invention encompasses naturally occurring CRISPR repeat sequences as well as functional variants thereof and synthetic versions. Exemplary GenBank accession numbers of other CRISPR1 sequences include: CP000023, CP000024, DQ072985, DQ072986, DQ072987, DQ072988, DQ072989, DQ072990, DQ072991, DQ072992, DQ072993, DQ072994, DQ072995, DQ072996, DQ072997, DQ072998, DQ072999, DQ073000, DQ073001, DQ073002, DQ073003, DQ073004, DQ073005, DQ073006, DQ073007, DQ073008, and AAGS01000003.

A naturally occurring CRISPR repeat sequence typically has about 20 to about 40 base pairs (e.g., about 36 base pairs). A CRISPR repeat sequence of the invention may include CRISPR repeat regions of this size range or every numerical range or integer therebetween. Additionally, the CRISPR repeat sequence may be larger or smaller as long as it still functions in the CRISPR mechanism. The number of CRISPR repeats in an array will also vary. For instance, the CRISPR array should include at least two repeat sequences, at a minimum in order to flank a single spacer. The number of repeats may range in some embodiments from about 2 to about 250.

As used herein, a “target specific spacer sequence” is a non-repetitive nucleic sequence positioned between repeats (i.e., CRISPR repeats) of the CRISPR array. In some embodiments of the present invention, a target specific spacer sequence refers to the nucleic acid segment that is flanked by two CRISPR repeats. It has been found that CRISPR spacer sequences often have significant similarities to a variety of mobile DNA molecules (e.g., bacteriophages and plasmids). In some preferred embodiments, target specific spacer sequence are located between two identical repeat sequences. The target specific spacer sequence include target sequence and are the key to identifying the DNA for destruction. Thus, at least one strand of the target specific spacer sequence is homologous to the target nucleic acid or a transcription product thereof and the other strand is complementary. The homologous and complementary sequences in some instances are 100% homologous or complementary to the target sequences. In some embodiments the target specific spacer sequences are at least about 70%, 75%, 80%, 85%, 90%, 91%, 92%, 93%, 94%, 95%, 96%, 97%, 98% or 99% complementary to the target nucleic acid sequence or a transcription product thereof. The target sequence is part of any one or more of a DNA based transposon, bacteriophage nucleic acid, plasmid, and/or chromosome.

The size of the target nucleic acid sequence may vary. In some instances it is about 20 to about 40 base pairs in length. A target nucleic acid sequence of the invention may include target nucleic acid sequence of this size range or every numerical range or integer therebetween. Additionally, the target nucleic acid sequence may be larger or smaller as long as it still functions in the CRISPR mechanism. In some embodiments the target nucleic acid sequence is in a range of 29-33, 30-33, 29-32, 29-31, 29-30, 30-32, 30-31, 31-33 or 32-32 base pairs. In other embodiments the target nucleic acid sequence is 29, 30, 31, 32, or 33 base pairs in length. Similar to the CRISPR repeat sequence, the number of target nucleic acid sequences in an array will also vary. For instance, the CRISPR array should include at least one target nucleic acid sequence positioned between repeat sequences. The number of repeats may range in some embodiments from about 2 to about 250.

The CRISPR array may also include a discriminator sequence adjacent to the target specific spacer sequences. In some instances the discriminator sequence is 5′ of the target specific spacer sequences and in other instances it may be 3′ of the target specific spacer sequences. A discriminator sequence is a short nucleotide sequence that promotes or inhibits functioning of the CRISPR array, depending on the type of CRISPR system. An example of a discriminatory sequence of the invention is a PAM sequence. PAM sequences are typically 3 base pairs in length, for example: ATG, AAG, GAG, AGG, AAA, AAC, AAT, TAG, TTG, ATA, CAG, TGG, GTG, GGG, AND TAA. PAMs have been described before in Mojica et al, Microbiology, 2009 (ATG and AAG) as well as Westra et al, PLoS Genetics, 2013 (GAG and AGG). PAM sequences discovered as being functional according to the invention also include strong sequences (AAA, AAC, AAT, TAG, TTG, and ATA) as well as weaker (CAG, TGG, GTG, GGG, and TAA).

Currently there are three known CRISPR systems. Type I CRISPR systems include at least two sub-subtypes, Type I-E and Type I-F. A complete set of Cas proteins from either an E- and or an F-subtype system could be used interchangeably as the DDD actuator, for example. Type II CRISPR systems typically rely on a single Cas protein to provide the same functionality as the larger set of Cas components in my Type I-E design. Thus, these systems may be technically more simple to design. Both Type I and Type II systems function optimally with a discriminator sequence such as PAM. However, in Type I systems, the PAM is typically 5′ to the target DNA whereas in Type II systems the PAM is 3′ to the target sequence. Type II systems also may include an additional processing element: an RNA component (a “tracrRNA”) and a host-encoded (non-Cas) housekeeping enzyme (RNAse III) to process the CRISPR RNA more efficiently. Type III systems are similar to Type I systems in their Cas & CRISPR components. However, Type III systems typically don't function with PAM sequences. These Type III systems, instead have a small subset of target-adjacent sequences are inhibitory if included in the construct if the target is to be degraded. These inhibitory sequences may also be referred to as “anti-PAM” sequences. Although most of the systems are designed to destroy DNA, the Type III-B systems destroy RNA rather than DNA.

An example of the positioning of PAM with respect to the target specific spacer sequences is presented below.

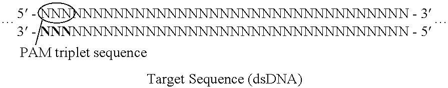

An important aspect of the nucleic acids of the invention is that they are each under the control of a regulatory element. The nucleic acid sequence of the actuator sequence and the nucleic acid sequence of the CRISPR array may share a single regulatory sequence. However in most embodiments, each of the two nucleic acids, even if present in a single vector, will each have a distinct regulatory element. A distinct regulatory element refers to its position such that it can control the expression of the nucleic acid sequence. The distinct regulatory elements may, however, be identical to one another or may be different from one another. The regulatory elements may also be activation elements or inhibitory elements. An activation element is a nucleic acid sequence that when presented in context with a nucleic acid to be expressed will cause expression of the nucleic acid in the presence of an activation signal. An inhibitory signal is a nucleic acid sequence that when presented in context with a nucleic acid to be expressed will cause expression of the nucleic acid unless an inhibitory signal is present. Each of the activation and inhibitory elements may be a promoter, such as a bacteriophage T7 promoter, sigma 70 promoter, sigma 54 promoter, lac promoter, etc.

Promoters may be constitutive or inducible. Examples of constitutive promoters include, without limitation, sigma 70 promoter, bla promoter, lacI. Promoter, etc.

TABLE 1
Commonly used inducible promoters

| | | Essential |
|---|---|---|
| | | regulatory |
| Name | Chemical inducer and/or repressor | gene(s) |
| ParaBAD | L-arabinose (ON) & glucose (OFF) | araC |
| (“PBAD”) | | |
| PrhaBAD | L-rhamnose (ON) & glucose (OFF) | rhaR & rhaS |
| Plac | lactose or IPTG (ON) & glucose (OFF) | lacI |
| Ptac | lactose or IPTG (ON) | lacI |
| Plux | acyl-homoserine lactone (ON) | luxR |
| Ptet | tetracycline or aTc (ON) | tetR |
| Psal | salycilate (ON) | nahR |
| Ptrp | tryptophan (OFF) | (NONE) |
| Ppho | phosphate (OFF) | phoB & phoR |

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

The regulatory elements may be in some instances tissue-specific. Tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some instances, the first regulatory element may be a first inducible promoter and the second regulatory element may be a second inducible promoter. In other instances the first regulatory element may be a first constitutive promoter and the second regulatory element may be a second constitutive promoter. In other instances either of the first or second regulatory element may be an inducible promoter while the other is a constitutive promoter. In other embodiments both the first and second regulatory elements may be activation elements or inhibitory elements. In other embodiments either of the first or second regulatory element may be an activation element and the other may be an inhibitory element.

When at least one of the regulatory elements is an activation signal, the system can be activated (partially or fully) with the use of an activation signal. An activation signal, as used herein, is a chemical (i.e. protein, nucleic acid, carbohydrate, small molecule, chemical compound etc.) or environmental signal (a compound or condition occurring in the environment, to which the nucleic acid will be exposed). Chemical signals include but are not limited to arabinose, glucose, lactose, IPTG, tetracycline, acyl-homoserine lactone, salycilate, tryptophan, phosphate. Environmental signals include but are not limited to temperature, glucose, pH, osmolarity, magnetic fields, electric fields, mechanical pressure, and radiation, including UV, gamma, visible light, infrared.

Thus the device of the invention is composed to two key components that are tightly regulated using a unique system of regulatory elements. The CRISPR array, a DNA sequence containing series of unique fragments interspersed between repeats of specific sequence encode the device's targeting information. The actuator component, for example, cas genes (i.e. cas3+casABCDE), encode the devices' enzymatic machinery sufficient for catalyzing DNA degradation. By placing these components under the artificial control of independent promoters, the degradation of specific target DNA can be controlled through the use of environmental or chemical signals at precise times and or under precise conditions.

This type of controlled regulatory system has important implications in biosecurity and biosafety. For instance, if a host containing a confidential or dangerous DNA sequence escapes into the environment or is stolen and attempts to grow in an environment lacking a signal molecules, the DNA will be destroyed, thus negating the threat. Additionally, artificial biological signal processing can be interrupted or activated at the control of the user. The DDD can be programmed to degrade synthetic plasmid DNA encoding specific sets of genetic regulatory elements when the host cell receive s a user-specified set of environmental or chemical cues. Thus, the host's downstream expression patterns can be altered in a controlled manner in response to changing conditions, such as those incurred during large scale industrial fermentations.

The unique combination of regulatory elements provides significant advantages to the manipulation of genetic material. Other prior methods for removing genetic material from a living prokaryotic host involve either inhibiting its replication (i.e. with temperature-sensitive or replication-incompetent origins) or selecting against/screening or stochastic loss. In either case, the genetic element is incompletely removed from the population at large. The DDD of the invention not only prevents propagation of a DNA sequence to progeny but also actively degrades these elements, lowering the total copy number of existing elements in the population gene pool. Additionally the DNA targeting is nucleotide-programmable. The targeting specificities of all other known prokaryotic nucleases capable of degrading DNA in vivo are encoded in their protein sequences. Thus, it is difficult if not impossible to readily alter the intended target sequence without extensive mutagenesis and screening. With the DDD, however, target specificity is directly encoded at the nucleotide level rather than the protein level. This allows for simple and fast retargeting of the device, either by resynthesizing a new synthetic DNA CRISPR array to match a valid protospacer within a target or by adding a small fragment of synthetic DNA sequence (matching protospacer to PA) to the intended target to match a preexisting CRISPR gene.

The target DNA may be deleted in its entirety in response to the methods of the invention. In some instances at least about 10%, 15%, 20%, 25%, 30%, 35%, 40%, 45%, or 50% of the target DNA is deleted from the host cell by administration of a DDD as described herein. In some embodiments, at least about 60%, 70%, or 80% by of the target DNA is deleted from the host cell by administration of a DDD of the invention. In some embodiments, at least about 85%, 90%, or 95% or more of the target DNA is deleted from the host cell by administration of a DDD as described herein. In some preferred embodiments 100% of the target DNA is deleted from the host cell by administration of a DDD.

In some aspects the invention relates to methods for using the DDD. For instance, a method for destroying target specific DNA in a living host cell is provided. The method involves contacting a living modified host cell having an exogenous nucleic acid actuator sequence under the control of a first regulatory element and an exogenous target specific nucleic acid CRISPR array under the control of a second regulatory element with a first regulatory signal, wherein the first regulatory signal induces the expression of the actuator sequence to produce an actuator protein, wherein the cell is exposed to a second regulatory signal and the second regulatory signal induces the production of a target specific DNA interference RNA, and wherein the actuator protein and the target specific DNA interference RNA destroy a target DNA in the host cell.

In some instances a plasmid comprising a nucleic acid sequence having a CRISPR array under the control of a regulatory element is provided. The plasmid can be used for insertion of target specific spacer sequences before use in the invention. The nucleic acid sequence having the CRISPR array has at least two palindromic repeat sequences with a spacer region positioned between the at least two palindromic repeat sequences. The spacer region includes at least two restriction enzyme sequences that can be used to insert the target specific spacer of interest. In some embodiments, a pre-spacer is present between the palindromic repeat sequences. In such cases, the restriction enzyme sequences can be used to first remove the pre-spacer sequence, then insert the target specific spacer. A nucleic acid sequence encoding a selectable marker may also be included for convenience, but is not essential.

Starting with the plasmid, a set of restriction enzymes can be used to cleave out the existing spacer DNA. A set of target specific spacer sequences having sticky ends associated with the restriction enzymes used can then be inserted. More than one spacer can be added at a time through the use of DNA manipulation and multiple restriction enzymes. Restriction enzymes or endonucleases cleave DNA with extremely high sequence specificity and due to this property they have become indispensable tools in molecular biology and molecular medicine. Over three thousand restriction endonucleases have been discovered and characterized from a wide variety of bacteria and archae. Comprehensive lists of their recognition sequences and cleavage sites can be found at REBASE. In some embodiments the restriction enzyme sequences are BsaI.

The plasmids of the invention include for example pBJC1544 (SEQ ID NO. 1) and homologs thereof. The nucleotide sequence of pBJC1544 is presented below. The promoter (“PJ23100”-Constitutive) sequence is underlined. CRISPR repeats are marked with double underline. The BsaI restriction sites are in capitals, italics and underlined. The capital ‘C’ is CRISPR promoter +1.

(SEQ ID NO. 1)

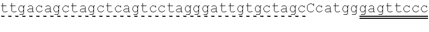

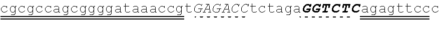

cgcgccagcggggataaaccgcagctcccattttcaaacccatcaagac
gcggtaccctcgagtctggtaaagaaaccgctgctgcgaaatttgaacg
ccagcacatggactcgtctactagcgcagcttaattaacctaggctgct
gccaccgcgcctgatgcggtattttctccttacgcatctgtgcggtatt
tcacaccgcatacgtcaaagcaaccatagtacgcgccctgtagcggcgc
attaagcgcggcgggtgtggtggttacgcgcagcgtgaccgctacactt
gccagcgccctagcgcccgctcctttcgctttcttcccttcctttctcg
ccacgttcgccggctttccccgtcaagctctaaatcgggggctcccttt
agggttccgatttagtgctttacggcacctcgaccccaaaaaacttgat
ttgggtgatggttcacgtagtgggccatcgccctgatagacggtttttc
gccctttgacgttggagtccacgttctttaatagtggactcttgttcca
aactggaacaacactcaaccctatctcgggctattcttttgatttataa
gggattttgccgatttcggcctattggttaaaaaatgagctgatttaac
aaaaatttaacgcgaattttaacaaaatattaacgtttacaattttatg
gtgcactctcagtacaatctgctctgatgccgcatagttaagccagccc
cgacacccgccaacacccgctgacgcgccctgacgggcttgtctgctcc
cggcatccgcttacagacaagctgtgaccgtctccgggagctgcatgtg
tcagaggttttcaccgtcatcaccgaaacgcgcgagacgaaagggcctc
gtgatacgcctatttttataggttaatgtcatgataataatggtttctt
agacgtcaggtggcacttttcggggaaatgtgcgcggaacccctatttg
tttatttttctaaatacattcaaatatgtatccgctcatgagacaataa
ccctgataaatgcttcaataatattgaaaaaggaagagtatggagaaaa
aaatcacgggatataccaccgttgatatatcccaatggcatcgtaaaga
acattttgaggcatttcagtcagttgctcaatgtacctataaccagacc
gttcagctggatattacggcctttttaaagaccgtaaagaaaaataagc
acaagttttatccggcctttattcacattcttgcccgcctgatgaacgc
tcacccggagtttcgtatggccatgaaagacggtgagctggtgatctgg
gatagtgttcacccttgttacaccgttttccatgagcaaactgaaacgt
tttcgtccctctggagtgaataccacgacgatttccggcagtttctcca
catatattcgcaagatgtggcgtgttacggtgaaaacctggcctatttc
cctaaagggtttattgagaatatgttttttgtctcagccaatccctggg
tgagtttcaccagttttgatttaaacgtggccaatatggacaacttctt
cgcccccgttttcacgatgggcaaatattatacgcaaggcgacaaggtg
ctgatgccgctggcgatccaggttcatcatgccgtttgtgatggcttcc
atgtcggccgcatgcttaatgaattacaacagtactgtgatgagtggca
gggcggggcgtaataataactgtcagaccaagtttactcatatatactt
tagattgatttaaaacttcatttttaatttaaaaggatctaggtgaaga
tcctttttgataatctcatgaccaaaatcccttaacgtgagttttcgtt
ccactgagcgtcagaccccgtagaaaagatcaaaggatcttcttgagat
cctttttttctgcgcgtaatctgctgcttgcaaacaaaaaaaccaccgc
taccagcggtggtttgtttgccggatcaagagctaccaactctttttcc
gaaggtaactggcttcagcagagcgcagataccaaatactgtccttcta
gtgtagccgtagttaggccaccacttcaagaactctgtagcaccgccta
catacctcgctctgctaatcctgttaccagtggctgctgccagtggcga
taagtcgtgtcttaccgggttggactcaagacgatagttaccggataag
gcgcagcggtcgggctgaacggggggttcgtgcacacagcccagcttgg
agcgaacgacctacaccgaactgagatacctacagcgtgagctatgaga
aagcgccacgcttcccgaagggagaaaggcggacaggtatccggtaagc
ggcagggtcggaacaggagagcgcacgagggagcttccagggggaaacg
cctggtatctttatagtcctgtcgggtttcgccacctctgacttgagcg
tcgatttttgtgatgctcgtcaggggggcggagcctatggaaaaacgcc
agcaacgcggcctttttacggttcctggccttttgctggccttttgctc
acatgttctttcctgcgttatcccctgattctgtggataaccgtattac
cgcctttgagtgagctgataccgctcgccgcagccgaacgaccgagcgc
agcgagtcagtgagcgaggaagcggaagagcgcccaatacgcaaaccgc
ctctccccgcgcgttggccgattcattaatgcagctggcacgacaggtt
tcccgactggaaagcgggggatctcgacgctctcccttatgcgactcct
ccattaccaaat.

The promoter of SEQ ID NO. 1 can readily be changed to create inducible rather than constitutive version. Examples are presented in the Figures. The Para/lac minimal sequence has the nucleic acid sequence of SEQ ID NO. 2. Capital ‘A’ is promoter +1.:

(SEQ ID NO. 2)
gaaaccaattgtccatattgcatcagacattgccgtcactgcgtctttt
actggctcttctcgctaaccaaaccggtaaccccgcttattaaaagcat
tctgtaacaaagcgggaccaaagccatgacaaaaacgcgtaacaaaagt
gtctataatcacggcagaaaagtccacattgattatttgcacggcgtca
cactttgctatgccatagcatttttatccataagattagcggatcctac
ctgacaattgtgagcgctcacaattactgtttctccAattgtgagcgct
cacaatt.

In one embodiment, a homologous sequence includes a nucleotide sequence that is at least about 85% or more homologous or identical to the nucleic acid of interest, e.g., at least 5%, 10%, 15%, 20%, 25%, 30%, 35%, 40%, 50% or more of the full length of the nucleic acid of interest). In some embodiments, the nucleotide sequence is at least about 90%, 91%, 92%, 93%, 94%, 95%, 96%, 97%, 98%, 99% or 100% homologous or identical to the nucleic acid of interest. In some embodiments, the nucleotide sequence is at least about 85%, e.g., is at least about 90%, 91%, 92%, 93%, 94%, 95%, 96%, 97%, 98%, 99% or 100% homologous or identical to the nucleic acid of interest.

Calculations of homology or sequence identity between sequences (the terms are used interchangeably herein) are performed as follows. To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The DDD created by the methods of the invention can be used to produce genetically modified organisms including modified hosts and host cells such as bacteria, yeast, mammals, plants, and other organisms through the deletion of target specific DNA. Genetically modified organisms may be used in research (e.g., as animal models of disease, as tools for understanding biological processes, and the role of target DNA in pathways etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), for deleting DNA that may be damaging to the environment in plasmids prior to disposal or if they are released accidentally and for other applications. These DDD also may be used as therapeutic compositions (e.g., for deleting deleterious DNA).

Thus, the DDD constructs of the invention may be expressed in vivo in a host organism or in vitro in a host cell. The host organism or host cell may be any organism or cell in which a DNA can be introduced. For example, organisms and cells according to the invention include prokaryotes and eukaryotes (i.e. yeast, plants). Prokaryotes include but are not limited to Cyanobacteria, Bacillus subtilis, E. coli, Clostridium, and Rhodococcus. Eukaryotes include, for instance, algae (Nannochloropsis), yeast such as, S. cerevisiae and P. pastoris, mammalian cells, such as for instance human cells, primary stem cell lineages, embryonic stem cells, adult stem cells, rodents, and plants. Thus, some aspects of this invention relate to engineering of a cell to integrate or express in a non-integrated manner RNA from the DDD components.

In some embodiments, the nucleic acid sequences of the DDDs are used to engineer a cell (e.g., a host cell). For example, in some embodiments, the CRISPR repeat(s) are inserted into the DNA of a cell (e.g., plasmid and/or genomic DNA of a host cell), using any suitable method known in the art. In additional embodiments, the nucleic acid sequences of the DDD are present in at least one construct, at least one plasmid, and/or at least one vector, etc. In further embodiments, these sequences are introduced into the cell using any suitable method known in the art.

As used herein “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. The nucleotide sequences are displayed herein in the conventional 5′-3′ orientation.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example using genetic recombination. Genetic recombination is the breaking and rejoining of DNA strands to form new molecules of DNA encoding a novel set of genetic information.

“Expression” refers to the process of converting genetic information of a polynucleotide into RNA through transcription, which is catalyzed by an enzyme, RNA polymerase, and into protein, through translation of mRNA on ribosomes.

As used herein the term “isolated nucleic acid molecule” refers to a nucleic acid that is not in its natural environment, for example a nucleic acid that has been (i) extracted and/or purified from a cell, for example, an algae, yeast, plant or mammalian cell by methods known in the art, for example, by alkaline lysis of the host cell and subsequent purification of the nucleic acid, for example, by a silica adsorption procedure; (ii) amplified in vitro, for example, by polymerase chain reaction (PCR); (iii) recombinantly produced by cloning, for example, a nucleic acid cloned into an expression vector; (iv) fragmented and size separated, for example, by enzymatic digest in vitro or by shearing and subsequent gel separation; or (v) synthesized by, for example, chemical synthesis. In some embodiments, the term “isolated nucleic acid molecule” refers to (vi) an nucleic acid that is chemically markedly different from any naturally occurring nucleic acid. In some embodiments, an isolated nucleic acid can readily be manipulated by recombinant DNA techniques well known in the art. Accordingly, a nucleic acid cloned into a vector, or a nucleic acid delivered to a host cell and integrated into the host genome is considered isolated but a nucleic acid in its native state in its natural host, for example, in the genome of the host, is not. An isolated nucleic acid may be substantially purified, but need not be. For example, a nucleic acid that is isolated within a cloning or expression vector is not pure in that it may comprise only a small percentage of the material in the cell in which it resides. Such a nucleic acid is isolated, however, as the term is used herein.

Methods to deliver expression vectors or expression constructs into cells, for example, into yeast cells, are well known to those of skill in the art. Nucleic acids, including expression vectors, can be delivered to prokaryotic and eukaryotic cells by various methods well known to those of skill in the relevant biological arts. Methods for the delivery of nucleic acids to a cell in accordance to some aspects of this invention, include, but are not limited to, different chemical, electrochemical and biological approaches, for example, heat shock transformation, electroporation, transfection, for example liposome-mediated transfection, DEAE-Dextran-mediated transfection or calcium phosphate transfection. In some embodiments, a nucleic acid construct, for example an expression construct comprising a fusion protein nucleic acid sequence, is introduced into the host cell using a vehicle, or vector, for transferring genetic material. Vectors for transferring genetic material to cells are well known to those of skill in the art and include, for example, plasmids, artificial chromosomes, and viral vectors. Methods for the construction of nucleic acid constructs, including expression constructs comprising constitutive or inducible heterologous promoters, knockout and knockdown constructs, as well as methods and vectors for the delivery of a nucleic acid or nucleic acid construct to a cell are well known to those of skill in the art, and are described, for example, in J. Sambrook and D. Russell, Molecular Cloning: A Laboratory Manual, Cold Spring Harbor Laboratory Press; 3rd edition (Jan. 15, 2001); David C. Amberg, Daniel J. Burke; and Jeffrey N. Strathern, Methods in Yeast Genetics: A Cold Spring Harbor Laboratory Course Manual, Cold Spring Harbor Laboratory Press (April 2005); John N. Abelson, Melvin I. Simon, Christine Guthrie, and Gerald R. Fink, Guide to Yeast Genetics and Molecular Biology, Part A, Volume 194 (Methods in Enzymology Series, 194), Academic Press (Mar. 11, 2004); Christine Guthrie and Gerald R. Fink, Guide to Yeast Genetics and Molecular and Cell Biology, Part B, Volume 350 (Methods in Enzymology, Vol 350), Academic Press; 1st edition (Jul. 2, 2002); Christine Guthrie and Gerald R. Fink, Guide to Yeast Genetics and Molecular and Cell Biology, Part C, Volume 351, Academic Press; 1st edition (Jul. 9, 2002); Gregory N. Stephanopoulos, Aristos A. Aristidou and Jens Nielsen, Metabolic Engineering: Principles and Methodologies, Academic Press; 1 edition (Oct. 16, 1998); and Christina Smolke, The Metabolic Pathway Engineering Handbook: Fundamentals, CRC Press; 1 edition (Jul. 28, 2009), all of which are incorporated by reference herein.

The nucleic acid sequences of the CRISPR array and the actuator are composed of nucleotides and may be DNA or RNA. In some embodiments, the nucleic acid is of genomic origin, while in other embodiments, it is of synthetic or recombinant origin. In some embodiments, the nucleic acid sequences are double-stranded or single-stranded whether representing the sense or antisense strand or combinations thereof. In some embodiments, nucleic acid sequences are prepared by use of recombinant DNA techniques (e.g., recombinant DNA).

In many cases the nucleic acids described herein having naturally occurring nucleotides and are not modified. In some instances, the nucleic acids may include non-naturally occurring nucleotides and/or substitutions, i.e. sugar or base substitutions or modifications. One or more substituted sugar moieties include, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of a nucleic acid; or a group for improving the pharmacodynamic properties of a nucleic acid and other substituents having similar properties. Similar modifications may also be made at other positions on the nucleic acid, particularly the 3′ position of the sugar on the 3′ terminal nucleotide and the 5′ position of 5′ terminal nucleotide. Nucleic acids may also have sugar mimetics such as cyclobutyls in place of the pentofuranosyl group.

Nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included.

In the context of the present disclosure, hybridization means base stacking and hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as the term is used in the art, refers to the capacity for precise pairing between two nucleotides. For example, if a nucleotide at a certain position of an nucleic acid is capable of hydrogen bonding with a nucleotide at the same position of a second nucleic acid, then the two nucleic acids are considered to be complementary to each other at that position. The nucleic acids are complementary to each other when a sufficient number of corresponding positions in each molecule are occupied by nucleotides that can hydrogen bond with each other through their bases. Thus, “specifically hybridizable” and “complementary” are terms which are used to indicate a sufficient degree of complementarity or precise pairing such that stable and specific binding occurs between the nucleic acids. 100% complementarity is not required.

In some instances the methods of the invention may be used for mammalian genome editing, particularly with Type I CRISPR, i.e., Type I(-E). For example the DDD may be assembled using a Type I CRISPR array and mammalian target specific spacers sequences. The actuator and CRISPR array are both under the control of regulatory elements that can control the timing and other conditions associated with the degradation. For instance, the DDD can be associated with a gene therapy vector and can be used to destroy the vector if destruction becomes necessary or desirable. Additionally the DDD may be used in a tumor system to selectively destroy oncogenes when appropriate. In most instances of the invention, however, the DDD is used in the context of non-mammalian hosts.

The invention also includes articles, which refers to any one or collection of components. In some embodiments the articles are kits. The articles include pharmaceutical, research or diagnostic grade compounds of the invention in one or more containers. The article may include instructions or labels promoting or describing the use or synthesis of the compounds of the invention. One kit includes one or more containers housing one or more components of a synthetic DDD selected from a nucleic acid sequence having an actuator sequence under the control of a first regulatory element and a nucleic acid sequence having a CRISPR array under the control of a second regulatory element, and instructions for delivering the components to a living cell.

In one embodiment, a kit comprises a single nucleic acid sequence having both the actuator sequence under the control of the first regulatory element and the CRISPR array under the control of the second regulatory element. In some instances the kit includes separate single nucleic acid sequences for the actuator sequence under the control of the first regulatory element and the CRISPR array under the control of the second regulatory element. Each nucleic acid may be in a plasmid. In some embodiments the nucleic acid sequence having the CRISPR array has at least two palindromic repeat sequences with a spacer region positioned between the at least two palindromic repeat sequences, wherein the spacer region includes at least two restriction enzyme sequences. The kit may also include a container housing a restriction enzyme, wherein the restriction enzyme is capable of cleaving at least one of the restriction enzyme sequences and optionally bacteriophage particles.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper administration of these agents. In certain embodiments agents in a kit may be in a pharmaceutical formulation and dosage suitable for a particular application and for a method of administration of the agents. In other embodiments the kit may include the components for adding the target spacer sequence to the plasmid described herein.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of to instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a host cell. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

The invention described herein can be utilized to remove genetic material from a living prokaryotic host cell. Currently available methods include temperature-sensitive or replication-incompetent origins or selecting against or screening for the stochastic loss of the genetic material. These methods all result in varying degrees of incomplete removal from the cell population at large.

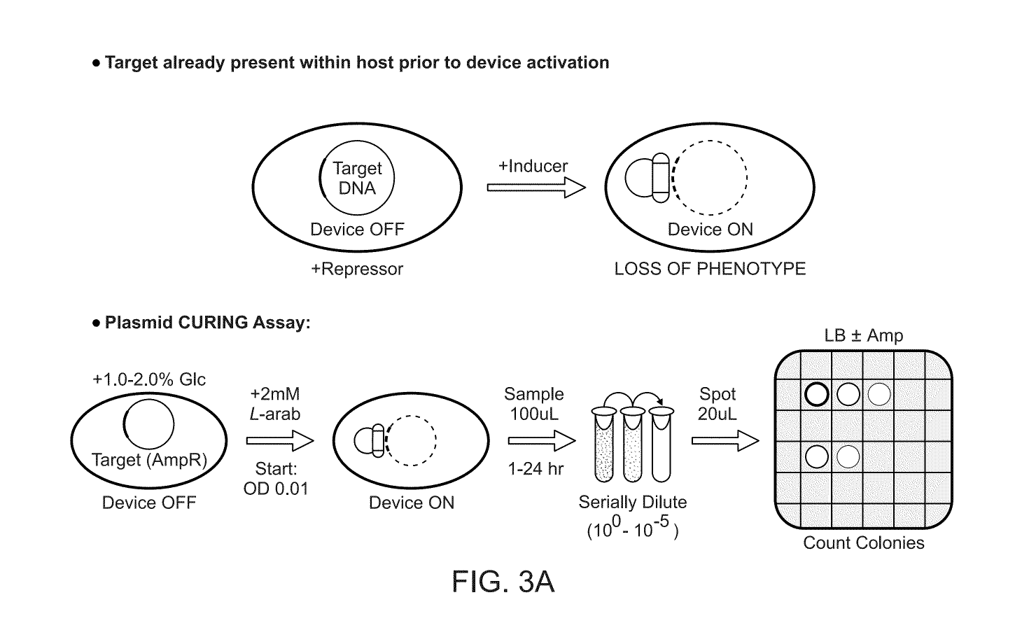

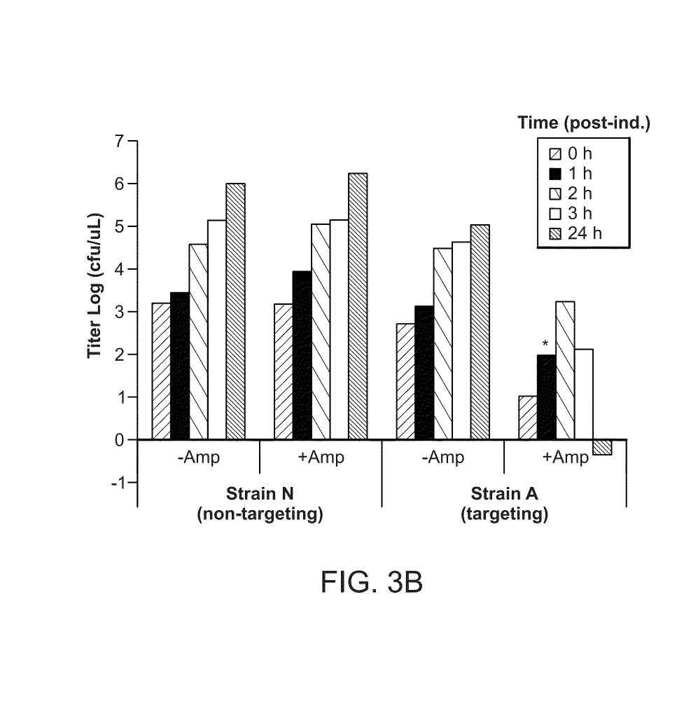

A method to assess the efficiency of the interference system is schematically represented in FIG. 3A. Briefly, a strain of E. coli encoding a CRISPR/cas system with a spacer sequence targeting the pBS (bla AmpR) plasmid, referred to as Strain A, and a strain of E. coli that does not target the pBS (bla AmpR) plasmid, referred to as Strain N, were transformed with the pBS (bla AmpR) target vector. The transformation reactions were plated on LB agar plates supplemented with ampicillin to select for cells that contain the plasmid. Single colonies were grown in liquid culture overnight at 37° C. in 2YT broth supplemented with ampicillin. Overnight cultures were diluted 1:100 into fresh 2YT broth without ampicillin and grown to early-log phage (OD600=0.025) and induced with 2 mM L-arabinose. Samples were then returned to 37° C. to grow overnight with periodic sample collection at 0, 1, 2, 3, and 24 hours.

At each indicated time point, samples were taken from the growing liquid cultures and serially diluted. Each dilution was plated onto two separate LB agar plates, one supplemented with ampicillin and one without ampicillin. Plates were incubated overnight at 37° at which point total number of colonies were enumerated from the LB plates without ampicillin. Similarly, colonies were enumerated from the LB plates supplemented with ampicillin to determine the population that retained the pBS plasmid and as a result, the ampicillin resistant phenotype.

As demonstrated in FIG. 3B, for Strain N, the E. coli strain that did not target the pBS (bla AmpR) plasmid, the total number ampicillin-resistant cells increased in parallel with the total cell count over the time course. Additionally at the 24-hour time point, 100% of the cells had retained the ampicillin-resistant phenotype indicating maintenance of the pBS plasmid. For Strain A, the E. coli strain that targeted the (pBS bla AmpR) plasmid, while the total number of cells did increase over time, the relative fraction of ampicillin-resistant cells dropped dramatically after 2-3 hours post inoculation. The loss of the ampicillin-resistant phenotype suggested that the synthetic CRISPR device in Strain A but not Strain N had destroyed its intended target DNA without killing the host cell.

### Example 2

In some applications of the described invention, the goal is to prevent or quickly eliminate entry of genetic material.

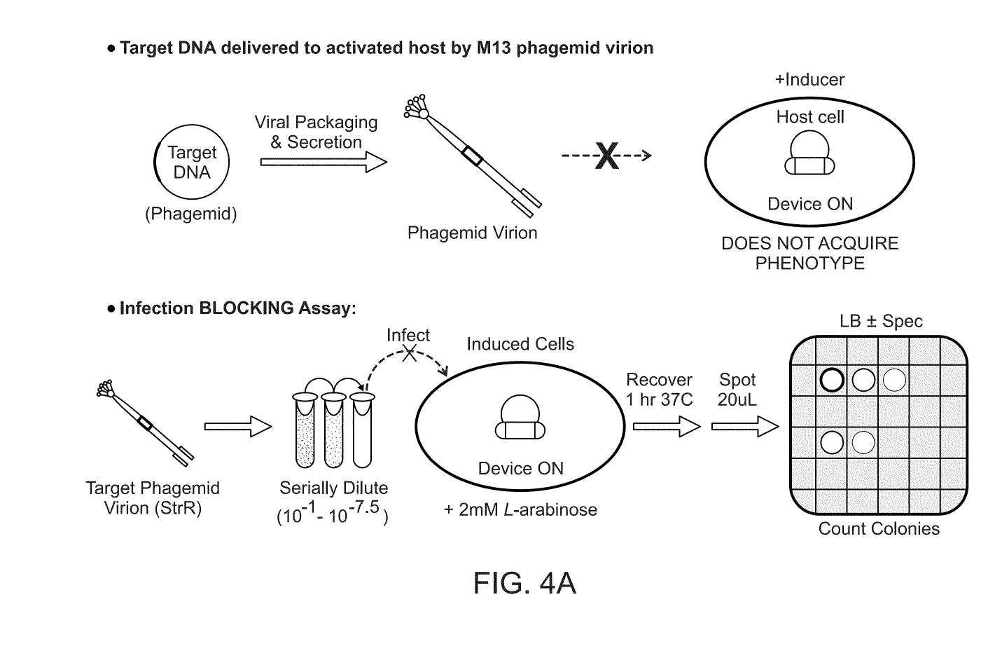

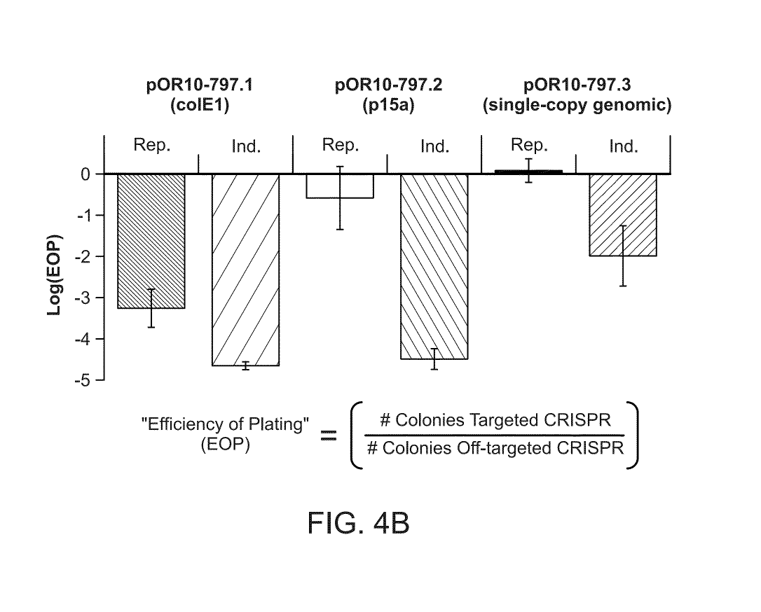

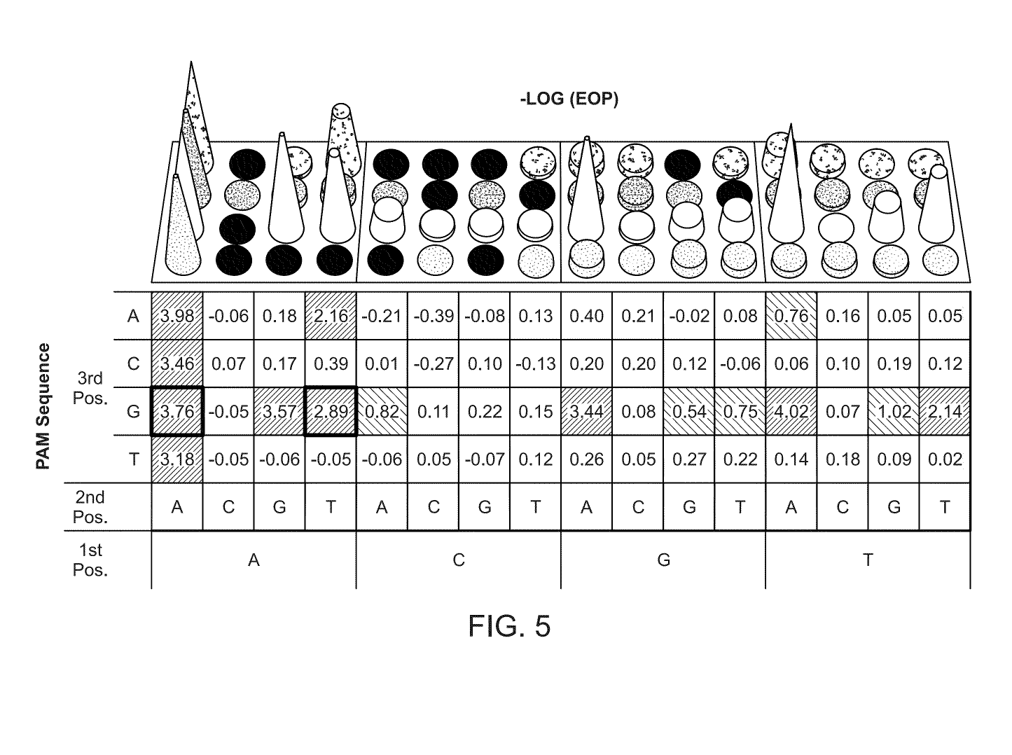

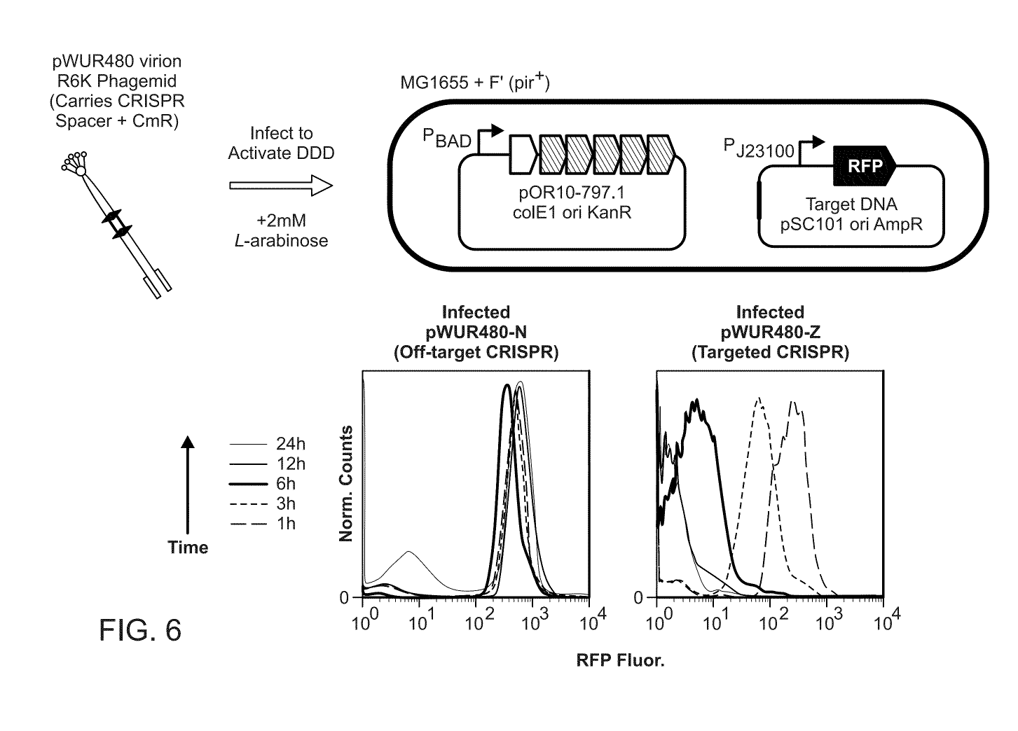

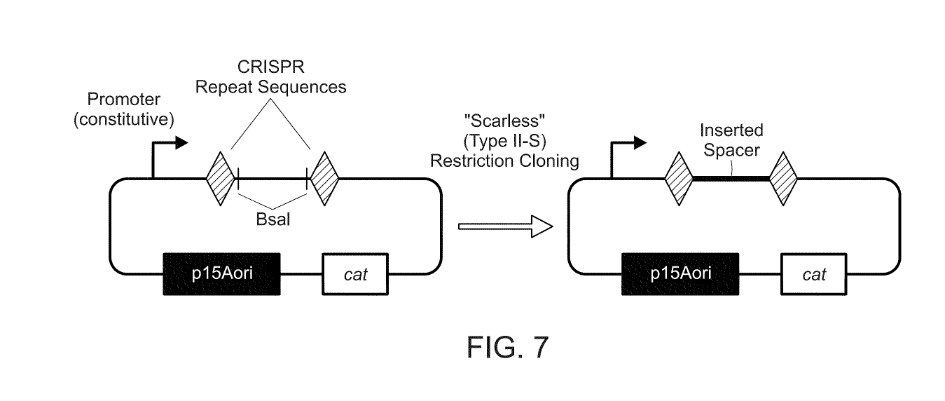

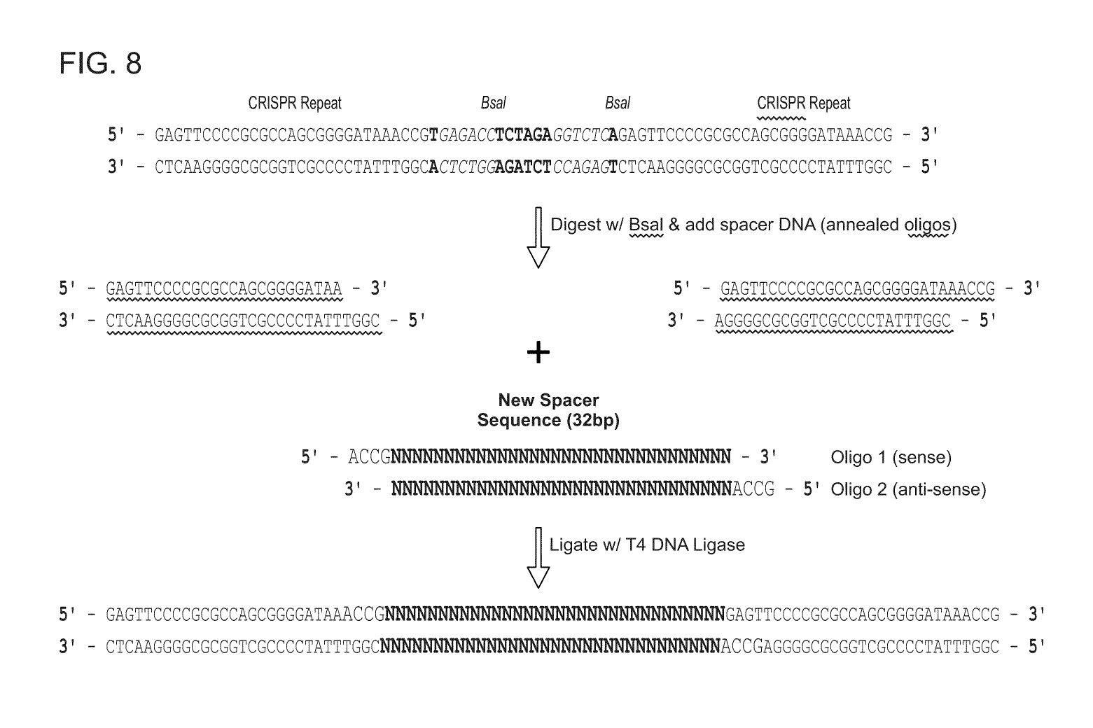

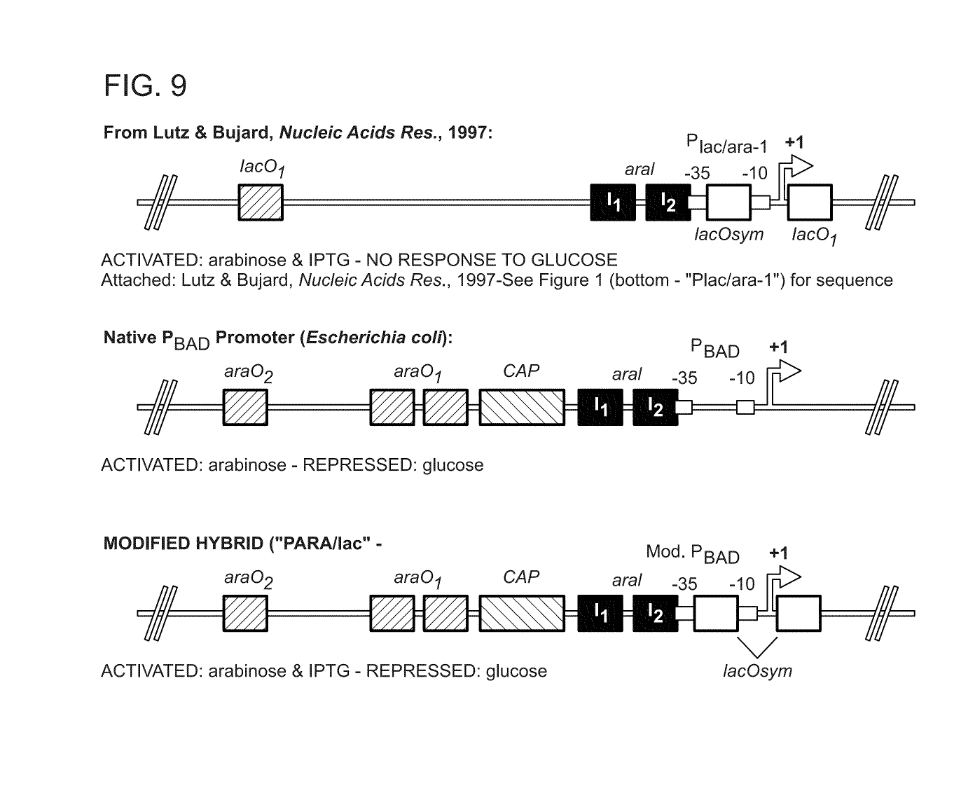

A method to assess the efficiency of the interference system in blocking target DNA entry is schematically represented in FIG. 4A. Briefly, a phagemid containing a target DNA sequence was packaged into M13 virions, and the virions were collected. A strain of E. coli encoding a CRISPR/cas system with a spacer sequence targeting a DNA sequence contained on the phagemid (pBS (bla AmpR) modified plasmid with SpecR), referred to as Strain A, and a strain of E. coli that does not target the DNA sequence contained on the phagemid or phage chromosome (pBS (bla AmpR) modified plasmid with SpecR), referred to as Strain N, were grown in LB broth early to mid-log phase (OD600). The cells were activated by supplementing the growth medium with 2 mM L-arabinose to induce the cas system. Activated cells were then transduced with the prepared M13 virions and incubated at 37° C. for one hour. Cells were plated on both LB agar plates and LB agar plates supplemented with spectinomycin. Plates were incubated overnight at 37° at which point total number of colonies were enumerated from the LB plates without spectinomycin. Similarly, colonies were enumerated from the LB plates supplemented with spectinomycin to determine the population that retained the phagemid and as a result, the spectinomycin resistant phenotype. The efficiency of plating (EOP) was calculated as the ratio of colonies with a targeted CRISPR to the number of colonies with an off-target CRISPR.

As demonstrated in FIG. 4B, for Strain M, the E. coli strain with the CRISPR that did not target the phagemid, had an EOP of XX. In contrast, the EOP for Strain Z, the E. coli strain with the CRISPR that targeted the phagemid was dramatically lower (105-fold reduction) The loss of efficiency of transduction suggested that the synthetic CRISPR device in the activated Strain Z but not Strain M had destroyed the incoming target DNA without killing the host cell.

### Example 3

The invention described herein can be utilized with many different promoters controlling the expression of the cas genes encoding the actuator complex. This allows for many different applications of the invention and aids in the value and utility of the system. As an example of this application of the described invention, the target specific spacers target sequences on a nucleic acid that is essential for replication or survival of the host organism, for example, the chromosome. The cas genes are regulated by a repressible promoter, for example a promoter repressed by glucose. In this case the culture of the organism in the presence of a repressor signal (glucose) expression of the cas genes and the target nucleic acid remains intact.

If the organism escapes, is stolen, or in any way not maintained in the presence of the repressor, the system will be activated, the cas genes expressed and the target nucleic acids degraded by CRISPR interference. As the target sequences are present on an essential nucleic acid molecule, the organism will not survive.

### Example 4

Plasmids and strains generated and used in Examples 1-3 were described.

| Plasmid | Description | Marker | ori |
|---|---|---|---|
| pWUR797 | P_{BAD} − cas3 + casABCDE | KANr | RSF |
| pWUR477 | P_{J23119}(constit.) − CRISPR N1-N8 | CMr | p15a |
| | (N1-N8 do not match any pBS | | |
| | proto-spacer sequence) | | |
| pWUR480 | P_{J23119}(constit.) − CRISPR A1-A5 | CMr | p15a |
| | (A5 identical to proto-spacer - PAM | | |
| | in bla A211E) | | |
| pBS | bla A211E | AMPr | pSC01 |
| | (Mutation inserts “AAG” PAM for | | |
| | Spacer A5) | | |
| pBS-2 | bla A211E | SPECr | |
| | (Mutation inserts “ATG” PAM for | | |
| | Spacer A5) | | |
| pWUR478.1 | P_{J23117}(constit.) − CRISPR M1-M2 | CMr | p15a |
| pWUR478.2 | P_{J23117}(constit.) − CRISPR M1-M2 | CMr | colE1 |
| pWUR481.1 | P_{J23117}(constit.) − CRISPR Z | CMr | p15a |
| pWUR481.2 | P_{J23117}(constit.) − CRISPR Z | CMr | p15a |
| pOR10-797.1 | P_{BAD} − cas3 + casABCDE | KANr | colE1 |
| pOR10-797.2 | P_{BAD} − cas3 + casABCDE | KANr | p15a |
| pOR10-797.3 | P_{BAD} − cas3 + casABCDE | KANr | R6K |
| Strain | | | |
| A | MG1655 F’ pWUR797 pWUR480 pBS | AMPr, | |
| | (DDD properly targets pBS for | KANr, | |
| | degradation) | CMr | |
| N | MG1655 F’ pWUR797 pWUR477 pBS | AMPr, | |
| | (DDD does not target pBS for | KANr, | |
| | degradation) | CMr | |
| M | MG1655 F’ pOR10-797.x pWUR478.x | | |
| | pBS-2 | | |
| Z | MG1655 F’ pOR10-797.x pS-2 | | |

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A synthetic DNA destruction device (DDD) comprising a nucleic acid sequence having an actuator sequence under the control of a first regulatory element and a nucleic acid sequence having a Clustered Regular Interspaced Short Palindromic Repeats (CRISPR) array under the control of a second regulatory element.

2. The synthetic DDD of claim 1, wherein the nucleic acid sequence having an actuator sequence and the nucleic acid sequence having a CRISPR array are linked.

3. The synthetic DDD of claim 1, wherein the actuator sequence encodes a DNA targeting/degradation protein.

4. The synthetic DDD of claim 1, wherein the actuator sequence encodes 2-10 DNA targeting/degradation proteins.

5. The synthetic DDD of claim 1, wherein the actuator sequence comprises a CRISPR-associated (cas) gene.

6. The synthetic DDD of claim 1, wherein the cas gene is selected from the group consisting of cas3 and casABCDE.

7. The synthetic DDD of claim 1, wherein the cas gene is six cas genes: cas3 and casABCDE.

8. The synthetic DDD of claim 5, wherein the cas gene is a single cas gene.

9. The synthetic DDD of claim 1, wherein the CRISPR array includes interspersed sets of target specific spacer sequences between palindromic repeat sequences.

10. The synthetic DDD of claim 9, wherein the interspersed sets of target specific spacer sequences are 29-33 base pairs in length.

11-19. (canceled)

20. The synthetic DDD of claim 9, wherein the target specific spacer sequences have an adjacent discriminator sequence.

21-22. (canceled)

23. The synthetic DDD of claim 20, wherein the adjacent discriminator sequence is a PAM sequence.

24-28. (canceled)

29. The synthetic DDD of claim 9, wherein the target sequence is part of any one or more of a DNA based transposon, bacteriophage nucleic acid, plasmid, and/or chromosome.

30. The synthetic DDD of claim 1, wherein the first regulatory element is a first inducible promoter.

31. The synthetic DDD of claim 1, wherein the second regulatory element is a second inducible promoter.

32. The synthetic DDD of claim 1, wherein the first regulatory element is an activation element that induces expression of the actuator sequence in response to one or more activation signals.

33. The synthetic DDD of claim 1, wherein the second regulatory element is a second activation element that induces the production of a DNA interference RNA from the CRISPR array in the presence of an activation signal.

34. The synthetic DDD of claim 32, wherein the activation signal is a chemical signal.

35. The synthetic DDD of claim 34, wherein the chemical signal is arabinose.

36. The synthetic DDD of claim 32, wherein the activation signal is an environmental signal.

37. The synthetic DDD of claim 1, wherein the first regulatory element is an inhibitory element that maintains the actuator in an inactive state by the presence of an inhibitory signal.

38. The synthetic DDD of claim 37, wherein the inhibitory signal is a chemical signal.

39. The synthetic DDD of claim 37, wherein the inhibitory signal is an environmental signal.

40. The synthetic DDD of claim 1, wherein the first regulatory element is an inhibitory element and an activation element.

41. The synthetic DDD of claim 1, wherein the second regulatory element is an inhibitory element and an activation element.

42. The synthetic DDD of claim 1, wherein the second regulatory element is an inhibitory element that maintains the CRISPR array in an inactive state by the presence of an inhibitory signal.

43-60. (canceled)

61. A method for destroying target specific DNA in a living host cell, comprising,
contacting a living modified host cell having an exogenous nucleic acid actuator sequence under the control of a first regulatory element and an exogenous target specific nucleic acid Clustered Regular Interspaced Short Palindromic Repeats (CRISPR) array under the control of a second regulatory element with a first regulatory signal, wherein the first regulatory signal induces the expression of the actuator sequence to produce an actuator protein, wherein the cell is exposed to a second regulatory signal and the second regulatory signal induces the production of a target specific DNA interference RNA, and wherein the actuator protein and the target specific DNA interference RNA destroy a target DNA in the host cell.

62-80. (canceled)

81. A plasmid comprising a nucleic acid sequence having a Clustered Regular Interspaced Short Palindromic Repeats (CRISPR) array under the control of a regulatory element, wherein the nucleic acid sequence having the CRISPR array has at least two palindromic repeat sequences with a spacer region positioned between the at least two palindromic repeat sequences, wherein the spacer region includes at least two restriction enzyme sequences and optionally a nucleic acid sequence encoding a selectable marker.

82-89. (canceled)
